(19) United States
(12) Patent Application Publication
(10) Pub. No.: US 2026/0071175 A1
(43) Pub. Date: Mar. 12, 2026

(54) Production Method and Culture Device of Cell Tissue

(71) Applicants: SHIMADZU CORPORATION, Kyoto-shi (JP); The University of Osaka, Suita-shi (JP)

(72) Inventors: Hikaru SHIBATA, Kyoto-shi (JP); Koji NUMATA, Kyoto-shi (JP); Yamato MAEDA, Kyoto-shi (JP); Rintaro YAMAMOTO, Kyoto-shi (JP); Shinsuke INOUE, Kyoto-shi (JP); Michiya MATSUSAKI, Suita-shi (JP)

(21) Appl. No.: 19/321,538

(22) Filed: Sep. 8, 2025

(30) Foreign Application Priority Data
Sep. 6, 2024 (JP) 2024-153669

(51) Int. Cl. C12N 5/00; C12M 1/00; C12M 1/33

(52) U.S. Cl. CPC C12N 5/0068; C12M 45/02; C12M 47/02

(57) ABSTRACT

The production method includes: linearly printing a plurality of cells between a first substrate and a second substrate in such a manner that both ends of the plurality of cells are supported on the first substrate and the second substrate, respectively; obtaining a plurality of cell tissues T by culturing the printed plurality of cells; filling a soluble liquid holding material between the first substrate and the second substrate; curing the filled holding material; separating each of the plurality of cell tissues T from at least one base material; and obtaining a plurality of blocks 50C by dividing the holding material along an extending direction of the plurality of cell tissues T held by the cured holding material. The obtaining of the plurality of blocks 50C includes housing the cured holding material in a dividing housing 30 provided with a plurality of blades 31.

### CROSS REFERENCE TO RELATED APPLICATIONS

This non-provisional application is based on Japanese Patent Application No. 2024-153669 filed on Sep. 6, 2024 with the Japan Patent Office, the entire contents of which are hereby incorporated by reference.

### BACKGROUND OF THE INVENTION

### Field of the Invention

The present disclosure relates to a production method and a culture device of a cell tissue.

### Description of the Background Art

Technology to construct cellular tissue by bioprinting has been studied. Research has been progressed to construct cell tissues by bioprinting and using the obtained cell tissues as edible stake meat or processed meat.

Michiya Matsusaki and others developed tendon-gel integrated bioprinting and demonstrated that fibrous cell tissues created by tendon-gel integrated bioprinting can be assembled to construct tissues such as artificial steaks in vitro (Michiya Matsusaki et al., “Engineered whole cut meat-like tissue by the assembly of cell fibers using tendon-gel integrated bioprinting”, Nature Communications, 12, 5059 (2021)). The tendon-gel integrated bioprinting disclosed in the above non-patent document is a method in which cells are printed on a gel that mimics a tendon and cultured with both ends of each cell joined to a support that mimics a tendon.

In order to realize such a culturing method, WO 2021/193980 discloses a method of culturing muscle cells by linearly arranging cells in such a manner that both ends of the cells are joined to a support.

WO 2021/193981 discloses a method of culturing cells by linearly printing a bioink that contains cells in a support bath, dissolving gels in the support bath after printing, removing solution in the support bath, and adding a culture solution to culture the cells.

### SUMMARY OF THE INVENTION

By culturing a cell with both ends of the cell supported on a substrate, the cell can be cultured with a tensile force generated between the substrate and the cell. The tensile force facilitates the formation of muscle fibers, muscle tissues or sarcomeric structures, which makes the resulting cell tissue more similar to an animal muscle.

As described above, although it is preferable to culture a cell with both ends of the cell supported on a substrate, it is necessary to remove the substrate when collecting the cell tissue after the culture. However, the cell tissue is very fragile. Therefore, there is a problem that the cell tissue may be damaged when the substrate is removed, which lowers the production efficiency.

It is an object of the present disclosure to provide a production method and a culture device of a cell tissue, which are capable of preventing damage to the cell tissue and have good production efficiency.

The production method of the present disclosure is a production method of a plurality of cell tissues. The production method includes: linearly printing a plurality of cells between a first substrate and a second substrate in such a manner that both ends of the plurality of cells are supported on the first substrate and the second substrate, respectively; obtaining the plurality of cell tissues by culturing the printed plurality of cells; filling, between the first substrate and the second substrate, a soluble liquid holding material that cures under a predetermined condition; curing the filled holding material; separating each of the plurality of cell tissues from at least one of the first substrate and the second substrate by cutting out the cured holding material from at least one of the first substrate and the second substrate; and obtaining a plurality of blocks by dividing the cured holding material along an extending direction of the plurality of cell tissues held by the cured holding material. The obtaining of the plurality of blocks includes housing the cured holding material in a dividing housing which is provided with a plurality of blades and is configured to divide and house the cured holding material.

The culture device of the present disclosure is a bioprinting culture device for culturing a plurality of cells to obtain a plurality of cell tissues. The culture device includes: a culture housing in which a first chamber with a first substrate for supporting the plurality of cells disposed therein, a storage chamber configured to store a culture solution, and a second chamber with a second substrate for supporting the plurality of cells disposed therein are provided in this order; a replacing unit that replaces the solution stored in the storage chamber with a soluble liquid holding material that cures under a predetermined condition; and a separating unit. The plurality of cells are linearly printed from the first chamber toward the second chamber. The separating unit is configured to separate each of the plurality of cell tissues stored in the storage chamber from at least one of the first substrate disposed in the first chamber and the second substrate disposed in the second chamber. The culture device further includes a dividing unit that divides the holding material along an extending direction of the plurality of cell tissues held by the holding material to obtain a plurality of blocks.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a schematic view illustrating a production method up to a culturing step.

FIG. 2 is a schematic view illustrating the other steps subsequent to the culturing step of the production method.

FIG. 3 is a diagram schematically illustrating a structure of a culture device according to a first embodiment.

FIG. 4 is a plan view of a wall viewed from a normal direction of a flat surface.

FIG. 5 is a view illustrating a printing step and a separating step using the culture device.

FIG. 6 is a plan view illustrating a flat surface of a wall according to a modification.

FIG. 7 is a diagram schematically illustrating a structure of a culture device according to a modification.

FIG. 8 is a schematic diagram illustrating a production method according to a second embodiment.

FIG. 9 is a view illustrating a holding material after a separating step according to a third embodiment.

FIG. 10 is a view illustrating the holding material after the separating step according to the third embodiment.

FIG. 11 is a diagram illustrating a dividing step according to the third embodiment.

FIG. 12 is a diagram illustrating the dividing step according to the third embodiment.

FIG. 13 is a diagram illustrating a dissolving step according to the third embodiment.

FIG. 14 is a schematic diagram illustrating a production method according to a fourth embodiment.

FIG. 15 is a diagram illustrating a dissolving step according to a modification of the fourth embodiment.

FIG. 16 is a diagram illustrating a dissolving step according to a modification of the fourth embodiment.

FIG. 17 is a diagram illustrating a dissolving step according to a modification of the fourth embodiment.

Hereinafter, embodiments of the present disclosure will be described in detail with reference to the drawings. In the drawings, the same or corresponding parts are denoted by the same reference numerals, and the description thereof will not be repeated.

### FIRST EMBODIMENT

### [Production Method of Cell Tissue]

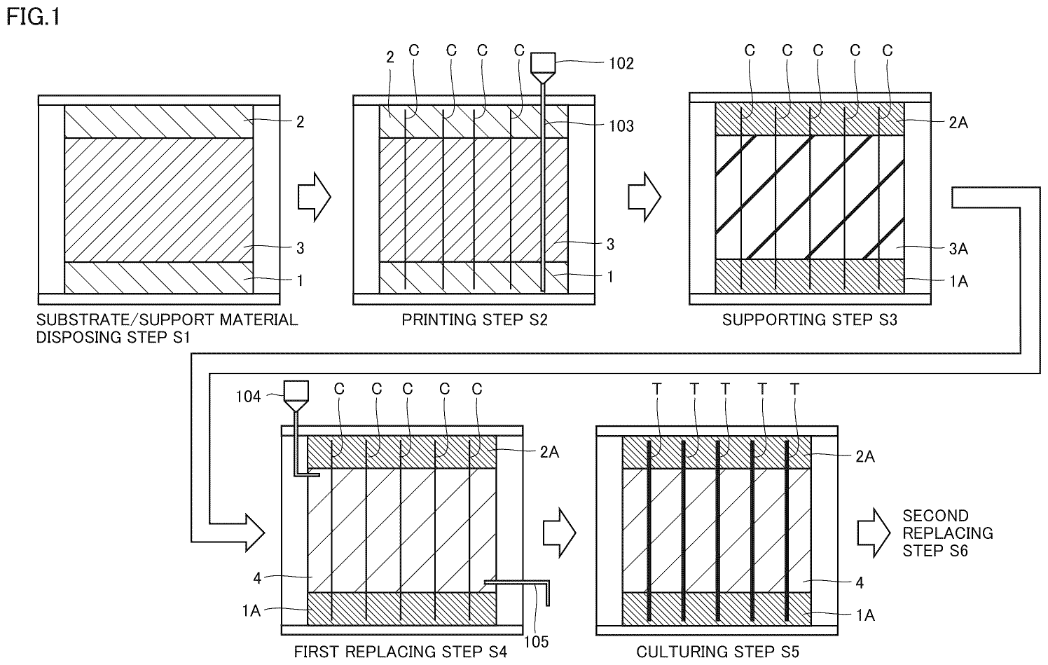

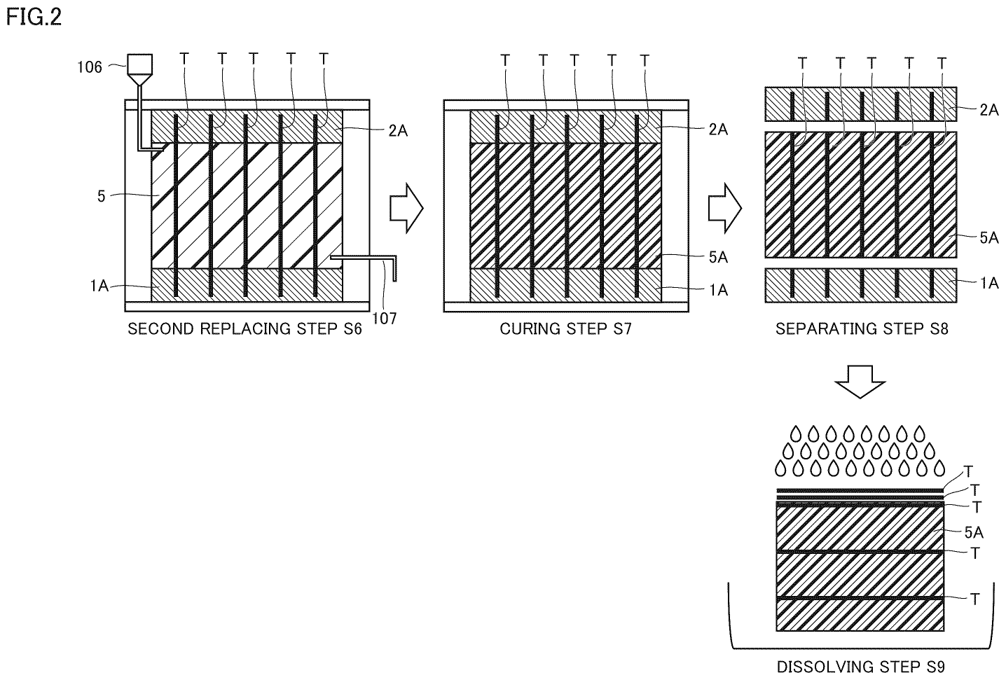

A production method will be described with reference to FIGS. 1 and 2. FIG. 1 is a schematic view illustrating a production method up to a culturing step. FIG. 2 is a schematic view illustrating the other steps subsequent to the culturing step in the production method.

The cell tissue is obtained by culturing a cell linearly printed. The cell is not particularly limited as long as it is linearly printed and cultured to form a fibrous cell tissue. The cell may be, for example, a cell derived from an animal, a cell derived from a human, or a cell derived from an animal other than a human. The cell used in the present embodiment is, for example, a skeletal muscle cell. The skeletal muscle cell may be a muscle-derived cell or a stem cell-derived cell. As another example, the cell used in the present embodiment is a fat cell. As yet another example, the cell used in the present embodiment is a cell that makes up a blood vessel. The cell tissue is used to constitute, for example, a fibrous muscle, a fibrous fat, or a fibrous blood vessel.

With reference to FIGS. 1 and 2, the production method according to the present embodiment includes a substrate/support material disposing step S1, a printing step S2, a supporting step S3, a first replacing step S4, a culturing step S5, a second replacing step S6, a curing step S7, a separating step S8, and a dissolving step S9.

### (Substrate/Support Material Disposing Step S1)

A substrate 1, a support material 3, and a substrate 2 are disposed in a culture device in this order. In other words, support material 3 is sandwiched between substrate 1 and substrate 2.

Substrates 1 and 2 may be any substrate that binds to a cell to be printed in the printing step S2 to support the cell, and may be made of a material appropriately selected by those skilled in the art. Substrates 1 and 2 may be either a solid or a liquid. The solid state includes a gel state. The physical properties of substrates 1 and 2 are not particularly limited, and for example, substrates 1 and 2 may have such a physical property that the state thereof changes under a predetermined condition or may such a physical property that the state thereof does not change under a predetermined condition. The predetermined condition is, for example, temperature, pressure, electrical stimulus, pH, or the like. Substrates 1 and 2 are, for example, collagen or collagen nanofibers. The type of collagen is not particularly limited. For example, substrates 1 and 2 may be formed from a plurality of different types of collagen. The composition of substrate 1 and the composition of substrate 2 may be different from each other. In the present embodiment, substrates 1 and 2 will be described as collagen nanofibers.

Support material 3 is made of a dissolvable material. For example, support material 3 is a solution obtained by dissolving a macromolecular substance such as gelatin, agar, or gellan gum in an aqueous solvent. In this case, in the substrate/support material disposing step S1, support material 3 may be in a gel form or a sol form. Support material 3 may have a thixotropy property, which means that when a force is applied, the viscosity thereof decreases and becomes a liquid, and when the force is removed, the viscosity thereof gradually recovers. Support material 3 may be, for example, a mixed solution prepared by pulverizing a gelled sample and dispersing the pulverized sample in a solvent such as a liquid medium. The gelled sample is prepared, for example, by dissolving a macromolecular substance such as gelatin, agar, or gellan gum in an aqueous solvent to form a gel. In the substrate/support material disposing step S1, support material 3 is described as a mixed solution in which a sample obtained by gelling a gelatin solution is pulverized and dispersed in a liquid medium.

In the present embodiment, as an example, after substrate 1 is disposed in liquid form, substrate 1 is partially cured to increase the viscosity, support material 3 made of a mixed solution which is obtained by dispersing a gelled sample in a solvent is disposed on substrate 1. Thereafter, substrate 2 is disposed in liquid form on support material 3. The method of disposing each sample is not particularly limited.

### (Printing Step S2)

The printing step S2 is a step of linearly printing cells C. At this time, cells C are printed in such a manner that one end of each cell C is disposed on substrate 1 and the other end thereof is disposed on substrate 2. For example, cells C are printed by the following method. A bioink containing cells C is filled into a syringe 102, the tip of which is attached with a nozzle 103. Thereafter, nozzle 103 is inserted from substrate 2 toward substrate 1 until the tip of nozzle 103 is located in substrate 1. Cells C are linearly printed from substrate 1 toward substrate 2 by ejecting the bioink from nozzle 103 while moving the tip of nozzle 103 in the direction from substrate 1 toward substrate 2.

The printing of cells is performed in support material 3. When support material 3 has a thixotropy property, the viscosity thereof decreases due to a stress caused by the movement of nozzle 103 and the ejection of the bioink during the printing, and the viscosity thereof recovers after the printing. Since the viscosity decreases during the printing, the nozzle can be easily moved and the bioink can be easily ejected. On the other hand, since the viscosity recovers and increases after the printing, support material 3 maintains the shape of the printed cells C and protects the printed cells C. When support material 3 is a solution obtained by dissolving a macromolecular substance in an aqueous solvent, the printing may be performed when support material 3 is in a sol form, and support material 3 may be turned into a gel form after the printing.

The printing step S2 may be performed automatically or semi-automatically using a known three-dimensional bioprinting apparatus. Alternatively, a multi-nozzle dispenser having a plurality of nozzles may be used to print a plurality of cells at one time.

The number of cells C linearly printed in the printing step S2 is not particularly limited as long as it is 1 or more, and may be 2 or more, 10 or more, 100 or more, 1000 or more, or 10000 or more, which may be appropriately selected according to the size of the final product, the culture device, and the like.

The diameter of a line formed by printing cells C is not particularly limited, and may be appropriately selected depending on the type of cells C, the viscosity of the bioink, the cell tissue to be obtained, the diameter of the usable nozzle 103, or the like. For example, the diameter of the line may be 100 μm or less, 100 μm or more, 1 mm or less, 1 mm or more, 10 mm or less, 10 mm or more, 100 mm or less, or 100 mm or more.

In the present embodiment, a plurality of cells C may be printed linearly from substrate 1 toward substrate 2, and the extending directions of the plurality of cells C may be or may not be parallel to each other. In the present embodiment, the plurality of cells C are printed in such a manner that the extending directions of the plurality of cells C are parallel to each other.

### (Supporting Step S3)

The supporting step S3 is a step of curing substrates 1 and 2 into substrates 1A and 2A to support both ends of each cell C on substrates 1A and 2A, respectively. In FIGS. 1 and 2, in order to clearly show that the state of substrates 1 and 2 has been changed from the liquid state to the solid state, the liquid substrates are represented as substrates 1 and 2, and the solid substrates are represented as substrates 1A and 2A with a different reference numeral. The solid state includes a gel state, and curing substrates 1 and 2 means transforming substrates 1 and 2 into a state suitable for supporting both ends of each cell C.

Substrates 1A and 2A may have a reversible property, which means that the state of the substrate can change from a solid state to a liquid state, or may have an irreversible property, which means that the state of the substrate cannot change from a solid state to a liquid state.

When the state of substrates 1 and 2 gradually changes from a liquid state to a solid state, the printing step S2 and the supporting step S3 may be performed in parallel. In other words, substrates 1 and 2 may be cured while cells C are being printed. By performing the printing step S2 and the supporting step S3 in parallel, at least one cell C is linearly printed with both ends thereof supported on substrates 1 and 2, respectively.

The solid (gel) in support material 3 may be transferred to a liquid (sol) while substrates 1 and 2 are being cured. For example, in the case where substrates 1 and 2 do not dissolve under a condition where the solid (gel) in support material 3 is transferred to the liquid (sol), the solid (gel) in support material 3 can be transferred to the liquid (sol) while substrates 1 and 2 are being cured by controlling the culture device so as to satisfy the condition for transferring the solid (gel) in support material 3 to the liquid (sol) and the condition for curing substrates 1 and 2. By transferring the solid (gel) in support material 3 to the liquid (sol), support material 3 can be easily replaced with the culture solution in the following first replacement step S4. In FIG. 1, the support material that contains a solid (gel) is illustrated as support material 3, and the support material after being transferred to a liquid (sol) is illustrated as support material 3A with a different reference numeral.

In the present embodiment, substrates 1 and 2 are collagen nanofibers, and support material 3 is a mixed solution in which a sample obtained by gelling a gelatin solution is pulverized and dispersed in a liquid medium. By maintaining the temperature in the culture device lower than a temperature at which collagen does not denature and equal to or higher than a temperature at which gelatin in support material 3 is transferred to a sol, the gel in support material 3 can be transferred to a sol while substrates 1 and 2 are being cured. For example, the temperature in the culture device may be maintained less than about 40° C., at 25° C. or more, at 35° C. or more, or at 37° C. or more. If the temperature in the culture device is maintained in such a temperature range, gelatin in support material 3 is dissolved while substrates 1 and 2 are being cured. In consideration of the influence on the growth of cells C, the temperature in the culture device is preferably maintained at 30° C. to 40° C., more specifically at 37° C.

### (First Replacing Step S4)

The first replacing step S4 is a step of replacing support material 3A with a culture solution 4. For example, culture solution 4 is injected between substrates 1A and 2A from a syringe 104 to eject the sample (support material 3A) between substrates 1 and 2 from a waste solution port 105. At this time, support material 3A can be easily removed by transferring the solid (gel) in support material 3 to a liquid (sol) in advance in the supporting step S3.

Culture solution 4 may be appropriately selected by those skilled in the art according to the type of cells or the like. For example, culture solution 4 may contain a differentiation-inducing factor for inducing differentiation of cells.

Although the supporting step S3 and the step of dissolving support material 3A are performed in parallel, they may be performed independently. For example, the step of dissolving support material 3A may be performed after the supporting step S3, and then the first replacing step S4 may be performed.

### (Culturing Step S5)

The culturing step S5 is a step of culturing cells C in culture solution 4 to obtain cell tissues T. The culture conditions may be appropriately designed by those skilled in the art according to the type of cells and the type of cell tissues to be obtained. During the culturing step S5, the first replacing step S4 may be performed to change the culture solution.

### (Second Replacing Step S6)

With reference to FIG. 2, the second replacing step S6 is a step of replacing culture solution 4 with a holding material 5 that is a soluble liquid cured under a predetermined condition and filling holding material 5 between substrate 1 and substrate 2. For example, holding material 5 is injected between substrates 1A and 2A from a syringe 106 to eject culture solution 4 from a waste solution port 107.

Holding material 5 is made of a soluble material, and is treated as a dissolved liquid in the second replacing step S6. Holding material 5 is obtained by dissolving a macromolecular substance such as gelatin, agar, or gellan gum in an aqueous solvent. In the present embodiment, holding material 5 is obtained by dissolving gelatin in an aqueous solvent. It is preferable that holding material 5 has such a hardness that holding material 5 can hold cell tissues T when cured. For example, when gelatin having a jelly strength of 300 g is used, holding material 5 is a gelatin solution in which the gelatin content is adjusted to a range of 35 mg/mL to 45 mg/mL, and more specifically, a gelatin solution in which the gelatin content is adjusted to 40 mg/mL.

Support material 3 and holding material 5 are common in that each is a sample that contains a macromolecular substance. In the present embodiment, support material 3 is a solution in which a macromolecular substance is gelled and dispersed in a solvent, whereas holding material 5 is a solution in which a macromolecular substance is completely dissolved and converted into a sol.

Holding material 5 may be used as a culture solution to be used in the culturing step S5. For example, a macromolecular substance such as gelatin may be dissolved in culture solution 4, and the obtained solution may be used to culture cells C. In this case, it is not necessary to perform the second replacing step S6. In other words, the step of filling holding material 5 may be performed before the culturing step S5 or in parallel with the culturing step S5. In the second replacing step S6, culture solution 4 is replaced with holding material 5, but holding material 5 may be used to replace a solution different from culture solution 4. In other words, a pretreatment step of increasing the strength of cell tissues T may be performed, or a step of washing cell tissues T may be performed before the second replacing step S6.

### (Curing Step S7)

The curing step S7 is a step of curing holding material 5. In FIG. 2, in order to clearly show that the state of holding material 5 has been changed from the liquid state to the solid state, the liquid holding material is represented as holding material 5, and the solid holding material is represented as holding material 5A with a different reference numeral. For example, the temperature in the culture device is maintained at 20° C. or less, 10° C. or less, or 5° C. or less. Since the curing condition varies depending on the type of holding material 5, the curing condition is determined according to the type of holding material 5.

### (Separating Step S8)

The separating step S8 is a step of separating substrates 1A and 2A from cell tissues T by separating cured holding material 5A from the cured substrates 1A and 2A. In this step, cell tissues T are separated from substrates 1A and 2A while being protected by holding material 5A. Therefore, a tensile force is less likely to be applied to cell tissues T in the separating step, and damage to cell tissues T can be prevented. Thus, the collection rate of cell tissues T can be increased.

In the present embodiment, the separating step S8 may be performed by taking out the entire block including substrates 1A and 2A and holding material 5A from a culture device 100. Even by taking out the entire block, since cell tissues T are protected by holding material 5A, damage to cell tissues T can be prevented, and the collection rate of cell tissues T can be increased.

### (Dissolving Step S9)

In the dissolving step S9, holding material 5A is dissolved to collect cell tissue T. Since the dissolving condition varies depending on the type of holding material 5A, the dissolving condition is determined according to the type of holding material 5A. In one example, in the dissolving step S9, a holding material dissolving liquid is used to dissolve holding material 5A. The holding material dissolving liquid is appropriately selected based on the dissolving condition of holding material 5A. As the holding material dissolving liquid, water having a predetermined temperature, a solution having a predetermined pH, or the like may be used. More specifically, for example, when holding material 5A is dissolved by temperature, holding material 5A that contains cell tissues T may be dissolved by applying water or the like having a predetermined temperature to holding material 5A to collect cell tissues T. When holding material 5A is dissolved by pH, holding material 5A may be dissolved by applying a solution (for example, a buffer solution) having a predetermined pH to holding material 5A so as to collect cell tissues T.

In one example, when holding material 5A is made of gelatin, water having a temperature of less than 40° C., 25° C. or more, 35° C. or more, or 37° C. or more (hereinafter referred to as “warm water”) is applied to holding material 5A. In particular, since cell tissues T (i.e., the cell tissues constituting the fibrous muscle, fat and blood vessel) may be degenerated by heat of about 40° C. or more, it is preferable to dissolve gelatin with water of 37° C. When holding material 5A is made of gellan gum, a cationic solution such as a tris-hydrochloric acid buffer solution, a tris-maleic acid buffer solution, or a bis-tris-buffer solution is applied to holding material 5A. When holding material 5A is made of agar, water having a temperature of 80° C. or more is applied to holding material 5A.

In consideration of the influence on cell tissues T, holding material 5A is preferably made of a material that dissolves at a temperature of less than 40° C. or a material that dissolves at a pH of 6.8 to 7.2. However, if holding material 5 can be separated from cell tissues T at the same time as holding material 5A is dissolved, the influence on cell tissues T can be reduced. Therefore, holding material 5 is not limited to those that dissolve under the above-described conditions, but may also dissolve under conditions that affect cell tissues T.

In the dissolving step S9, instead of applying a holding material dissolving liquid to holding material 5A containing cell tissue T, holding material 5A containing cell tissue T may be immersed in a water tank (water bath) that contains the holding material dissolving liquid. The device configured to apply the “holding material dissolving liquid” or the “water bath that contains the holding material dissolving liquid” corresponds to a “dissolving unit” configured to dissolve holding material 5A containing cell tissue T. The device configured to apply the holding material dissolving liquid includes, for example, a container that contains the holding material dissolving liquid, a liquid application unit configured to apply the holding material dissolving liquid contained in the container to holding material 5A, and a controller configured to control the liquid application unit.

In the present embodiment, a plurality of cells C are printed in the printing step S2 to obtain a plurality of cell tissues T. Each of the plurality of cells C is linearly printed from substrate 1 toward substrate 2. Therefore, the plurality of cell tissues T are held by holding material 5A in such a manner that the extending directions thereof are aligned with each other.

Therefore, as illustrated in FIG. 2, at the time of dissolving holding material 5A, it is preferable to dispose holding material 5A in such a manner that a surface on which holding material 5A is disposed and the extending direction of cell tissues T are aligned with each other. Thus, the plurality of cell tissues T can be collected in such a manner that the extending directions thereof are aligned with each other. As a result, in the step of assembling the collected cell tissues T, the step of aligning the fiber directions of cell tissues T becomes unnecessary.

Since the fibrous cell tissue is typically thin (for example, about 1 mm in diameter) and soft, when holding material 5A is immersed in a water bath containing a holding material dissolving liquid, in order to prevent cell tissues T from being drifted away by a flow caused by convection or agitation, which may scatter the extending directions of cell tissues T, it is preferable that holding material 5A is immersed in a holding material dissolving liquid that is not flowing.

According to the present embodiment, in the separating step S8, both of substrates 1A and 2A are separated from a plurality of cell tissues T. However, in the separating step S8, at least one of substrates 1A and 2A may be separated from the plurality of cell tissues T. When a material that does not dissolve in the dissolving step S9 is used as the substrate, since one end of each cell tissue T is held even after the dissolving step S9, the plurality of cell tissues T may be easily handled together.

### [Culture Device for Implementing Separating Step]

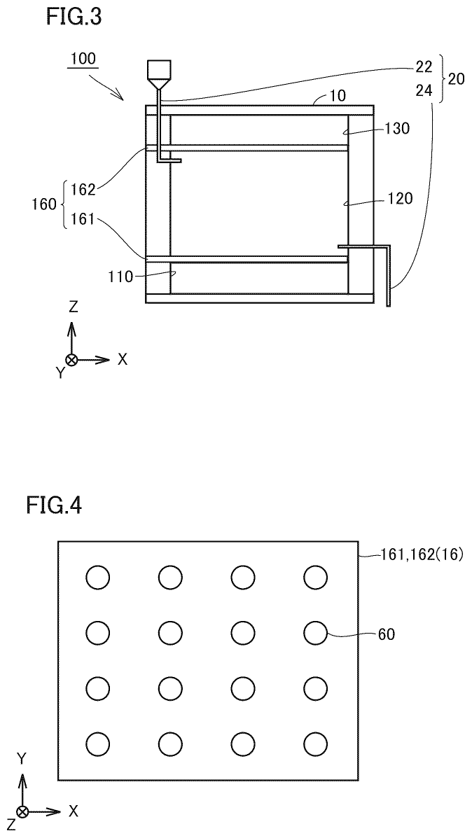

FIG. 3 is a diagram schematically illustrating a structure of a culture device 100 according to a first embodiment. Culture device 100 is a bioprinting culture device for culturing cells to obtain cell tissues. Culture device 100 includes a culture housing 10, a replacing unit 20, and a separating unit 160.

In culture housing 10, a first chamber 110 with substrate 1 disposed therein, a storage chamber 120 configured to store culture solution 4, and a second chamber 130 with substrate 2 disposed therein are provided in this order.

The substrate is disposed in each of first chamber 110 and second chamber 130. Culture device 100 may be configured in such a manner that the substrate is preliminarily disposed in each of first chamber 110 and second chamber 130, or may be configured in such a manner that the substrate is inserted into each of first chamber 110 and second chamber 130 later and disposed therein.

Replacing unit 20 replaces the sample (solution) filled in storage chamber 120 with another sample (solution). Specifically, replacing unit 20 includes an insertion port 22 for inserting a tube, a syringe, or the like to which a tube pump is attached, and a discharge port 24 for discharging the sample in storage chamber 120 to the outside of culture device 100. Insertion port 22 and discharge port 24 are each connected to storage chamber 120.

For example, in the first replacing step S4, replacing unit 20 replaces the sample filled in storage chamber 120 from support material 3A to culture solution 4. In addition, after cells C are cultured in the culturing step S5 and thereby cell tissues T are obtained, replacing unit 20 replaces the sample filled in storage chamber 120 from culture solution 4 to holding material 5 in the second replacing step S6.

Separating unit 160 is a device for separating cell tissues T obtained by culturing cells C in storage chamber 120 from the substrate. Separating unit 160 includes a first wall 161 and a second wall 162. First wall 161 is a wall that partitions first chamber 110 and storage chamber 120 from each other, and is disposed in culture housing 10. Second wall 162 is a wall that partitions storage chamber 120 and second chamber 130 from each other, and is disposed in culture housing 10. First wall 161 is disposed in culture housing 10 in such a manner that a flat surface of first wall 161 faces first chamber 110. Second wall 162 is disposed in culture housing 10 in such a manner that a flat surface of second wall 162 faces second chamber 130.

Both first wall 161 and second wall 162 are slidable. More specifically, first wall 161 is slidable along a flat surface of first wall 161. Second wall 162 is slidable along a flat surface of second wall 162. In FIG. 3, a sliding direction is defined as the X axis, a normal direction of the flat surface is defined as the Z axis, and an axis orthogonal to the X axis and the Z axis is defined as the Y axis. In the present embodiment, first wall 161 and second wall 162 are disposed in parallel to each other in culture housing 10, and have the same sliding direction. The sliding direction of first wall 161 and the sliding direction of second wall 162 may be different from each other.

When a substrate is disposed in each of first chamber 110 and second chamber 130, it can be said that first wall 161 is disposed in culture device 100 in such a manner that the flat surface thereof faces the substrate. Similarly, it can be said that second wall 162 is disposed in culture device 100 in such a manner that the flat surface thereof faces the substrate.

FIG. 4 is a plan view of a wall viewed from a normal direction of the flat surface. Hereinafter, first wall 161 and second wall 162 are collectively referred to as “wall 16”. An opening 60 is formed in wall 16. The plan view illustrated in FIG. 4 is obtained by viewing wall 16 from the normal direction (Z-axis direction) of the flat surface of wall 16.

At least one opening 60 is formed in wall 16. The number of openings 60 is not particularly limited, and the number may be 2 or more, 10 or more, 100 or more, 1000 or more, or 10000 or more. Since at least one opening 60 is formed in first wall 161 and second wall 162, the printing step S2 can be performed without removing first wall 161 and second wall 162.

First wall 161 and second wall 162 each may be a plate-shaped member in which opening 60 is not formed. In this case, after substrates 1 and 2 and support material 3 are disposed in culture device 100, the printing step may be performed by sliding first wall 161 and second wall 162 to eliminate the partition between storage chamber 120 and first chamber 110 and the partition between storage chamber 120 and second chamber 130. Thereafter, in the separating step, cell tissues T may be separated from substrates 1 and 2 by sliding first wall 161 and second wall 162 between storage chamber 120 and first and second chambers 110, 130.

The size of opening 60 should be large enough to allow a nozzle configured to eject the bioink containing the cells to pass through. When opening 60 is circular, the diameter of opening 60 may be 100 μm or less, 100 μm or more, 1 mm or less, 1 mm or more, 10 mm or less, 10 mm or more, 100 mm or less, or 100 mm or more.

First wall 161 and second wall 162 are preferably disposed in culture device 100 in such a manner that opening 60 formed in first wall 161 and opening 60 formed in second wall 162 face each other.

As illustrated in FIG. 4, wall 16 may be formed with a plurality of openings 60 arranged in two-dimensions. Cells C are printed in such a manner that at least one cell C passes through each of the plurality of openings 60 arranged in two-dimensions. As described above, since wall 16 is formed with a plurality of openings 60 arranged in two-dimensions, each cell tissue T can be easily handled after cell tissues T are separated from substrates 1 and 2.

The plurality of openings 60 may be arranged in two directions intersecting each other. The intersection angle is 30° or less, 30° or more, 45° or less, 45° or more, 90° or less, or 90° or more. In the example illustrated in FIG. 4, the plurality of openings 60 are arranged in two directions orthogonal to each other. The plurality of openings 60 may be arranged in a zigzag grid pattern.

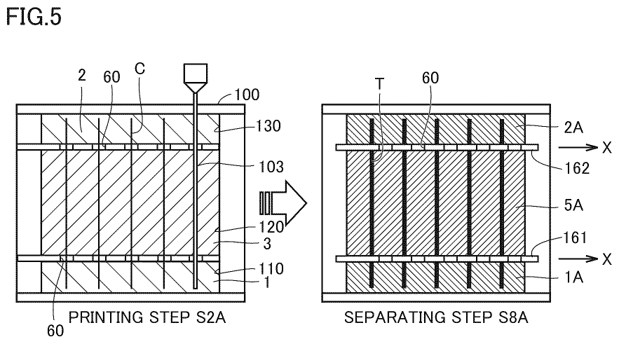

FIG. 5 is a view illustrating a printing step and a separating step using the culture device. In the printing step S2A, cells C can be linearly arranged in culture device 100 in such a manner that both ends of cells C are located in first chamber 110 and second chamber 130, respectively, by moving nozzle 103 to eject the bioink containing cells C as described below. First, nozzle 103 is disposed to pass through opening 60 of second wall 162 and opening 60 of first wall 161. Next, nozzle 103 is moved so that the tip of nozzle 103 passes through opening 60 of first wall 161 and opening 60 of second wall 162 while the bioink is being ejected from nozzle 103. Thus, cells C can be linearly arranged to pass through opening 60 of second wall 162 from opening 60 of first wall 161. As a result, cells C are linearly arranged from substrate 1 disposed in first chamber 110 toward substrate 2 disposed in second chamber 130. Thereafter, both ends of linear cells C can be supported on substrates 1 and 2 by curing substrates 1 and 2.

Next, in the separating step S8A, first wall 161 and second wall 162 are slid in the X-axis direction along the corresponding flat surface. Thus, cell tissues T, which are arranged to pass through opening 60, are cut off by an edge of opening 60. As a result, cell tissues T are separated from substrates 1A and 2A.

Thus, since cell tissues T can be separated from substrates 1 and 2 simply by sliding first wall 161 and second wall 162, respectively, the separating operation can be easily performed.

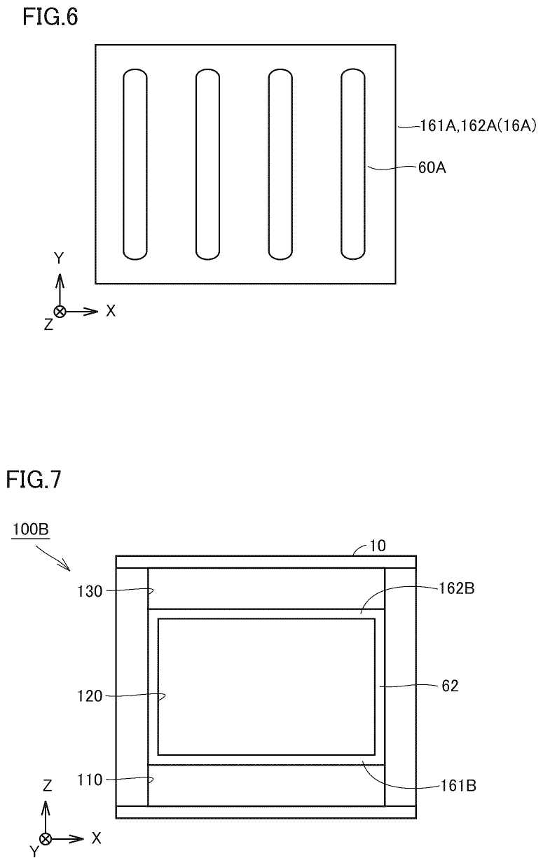

The arrangement pattern and shape of opening 60 are not limited to those illustrated in FIG. 4. FIG. 6 is a plan view of a wall viewed from a flat surface thereof according to a modification. A wall 16A is formed with a plurality of openings 60A, each of which has an elongated shape in which a length along the X-axis direction (sliding direction) is shorter than a length along the Y-axis direction orthogonal to the X-axis direction (sliding direction).

When each opening 60A has an elongated shape, as compared with the case where a plurality of openings 60 are formed in the column direction, it is easy to perform alignment when a plurality of nozzles of a multi-nozzle dispenser, for example, are inserted into culture device 100 at a time. Since the length along the X-axis direction is shorter than the length along the Y-axis direction, the distance from cell tissues T to the edge of each opening 60A can be reduced. Thus, it is possible to shorten the distance to slide wall 16A to separate cell tissues T from the substrates.

In addition, although it is described that storage chamber 120 is divided into first chamber 110 and second chamber 130 by first wall 161 and second wall 162, storage chamber 120 may be formed by arranging a box in culture housing 10. FIG. 7 is a diagram schematically illustrating a structure of a culture device 100B according to a modification. Culture device 100B may include a box 62 disposed in such a manner that the box is sandwiched between predefined spaces (first chamber 110 and second chamber 130) in culture housing 10. The inner space of box 62 corresponds to storage chamber 120. A first wall 161B which forms a boundary between first chamber 110 and box 62 is formed with an opening, and a second wall 162B which forms a boundary between second chamber 130 and box 62 is formed with an opening. The shape of the opening is not particularly limited, and for example, the opening having the shape illustrated in FIG. 4 or FIG. 6 may be formed in first wall 161B and second wall 162B.

Each of first wall 161B and second wall 162B is slidable along the flat surface of the wall, and at least one of first wall 161B and second wall 162B is openable in the Z-axis direction perpendicular to the wall.

Thus, by forming at least one of first wall 161B and second wall 162B as an openable and closable lid, it is possible to easily take out the holding material containing the cell tissues from culture device 100B after the cell tissues are separated from the substrates. Thus, each cell tissue can be easily handled after the cell tissues are separated from the substrates.

Although first wall 161 and second wall 162 that partition a respective space in culture housing 10 are defined as separating unit 160, the configuration of separating unit 160 is not limited thereto. For example, separating unit 160 may be realized by providing an opening in a side surface of culture housing 10 so that a flat plate can be inserted into storage chamber 120 in a direction (the X-axis direction or the Y-axis direction) orthogonal to the extending direction (the Z-axis direction) of cell tissues T. In this case, first wall 161 and second wall 162 may not be provided.

### SECOND EMBODIMENT

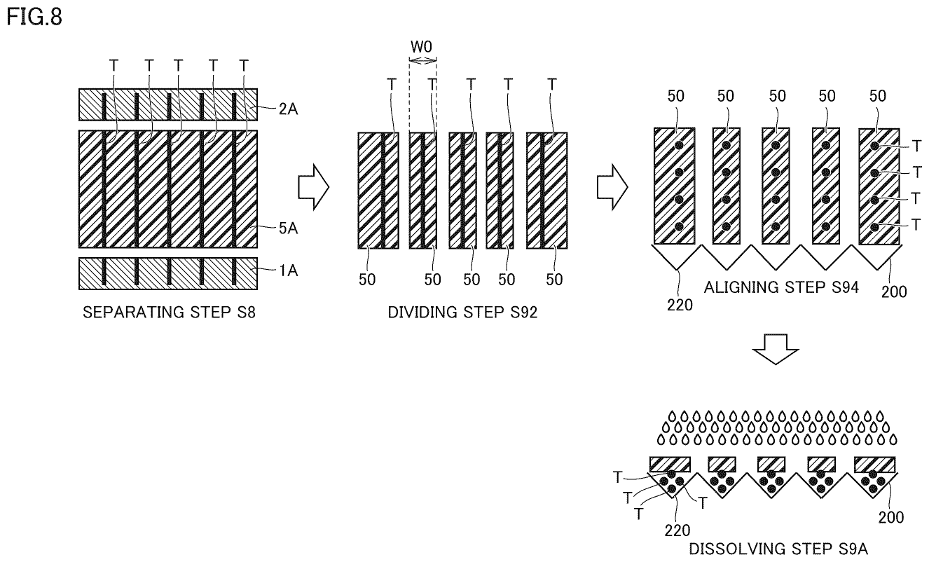

In the first embodiment, after cell tissues T are separated from substrates 1A and 2A, holding material 5A is dissolved in the dissolving step S9 as a large mass. However, before holding material 5A is dissolved, holding material 5A may be divided into a plurality of blocks and then the plurality of blocks may be dissolved. FIG. 8 is a schematic view illustrating a production method according to a second embodiment. Since the production method according to the second embodiment has the same steps up to the separating step S8 as the production method illustrated in FIGS. 1 and 2, only the steps after the separating step S8 are illustrated in FIG. 8.

The production method according to the second embodiment is different from the production method illustrated in FIGS. 1 and 2 in that the production method according to the modification further includes a dividing step S92 and an aligning step S94, and includes a dissolving step S9A instead of the dissolving step S9.

### (Dividing Step S92)

In the dividing step S92, cured holding material 5A is divided into a plurality of blocks 50 along the extending direction of cell tissues T. At least one block 50 of the plurality of blocks 50 contains at least one cell tissue T. The number of cell tissues T contained in each block 50 may be appropriately selected by those skilled in the art, and is determined according to, for example, subsequent processing steps. The number of blocks 50 may be two or more. For example, when a plurality of cells C are printed in two directions orthogonal to each other as illustrated in FIG. 4, holding material 5A may be divided into one row or two rows or more.

In a first example of the dividing step S92, holding material 5A is punched using a mold having a width corresponding to a width W0 of a desired block 50. In a second example, holding material 5A is cut by a cutting device (a cutter, a metal plate, or the like) at an interval corresponding to a desired width of the block 50. Each of the mold in the first example and the cutting device in the second example corresponds to an embodiment of a “dividing unit” that divides holding material 5A along an extending direction of the plurality of cell tissues T held by cured holding material 5A to obtain a plurality of blocks. Further, the width of the mold in the first example and the cutting interval in the second example correspond to an embodiment of “an interval between adjacent blades that divide the holding material in the dividing step”, which will be described later.

Since holding material 5A is divided into a plurality of blocks 50, it is possible to handle the cell tissues in each block 50; since the cell tissues are protected by cured holding material 5A constituting the plurality of blocks 50, it is easy to handle the cell tissues. In addition, one or a bundle of cell tissues T can be easily obtained by dissolving each block 50. In addition, in a case where a combination operation of combining a plurality of types of cell tissues T is performed to create an aggregate that mimics the structure of a specific tissue, it is easier to perform the combination operation in each block 50 than in a case where the combination operation is performed on each cell tissue T.

### (Aligning Step S94)

The aligning step S94 is a step of aligning the plurality of blocks 50. The plurality of blocks 50 are arranged so that the extending directions of cell tissues T are aligned with each other. In this case, each of the plurality of blocks 50 may be arranged on a net 200 having a wavy structure. When each block 50 is arranged on net 200 having a wavy structure, each block 50 may be disposed in a recess 220 so that the extending direction of recess 220 is aligned with the extending direction of cell tissues T included in each block 50. In the example of FIG. 8, since the plurality of blocks 50 are spaced apart from each other, the aligning step S94 also corresponds to an embodiment of a “spacing step” of spacing the plurality of blocks apart from each other. In addition, in the example of FIG. 8, the device that spaces the plurality of blocks 50 apart from each other corresponds to an embodiment of a “spacing unit”. By spacing the blocks 50 apart from each other, the dissolution rate can be increased as compared with the case where the blocks are not spaced apart from each other (which will be described later in detail).

### (Dissolving Step S9A)

The dissolving step S9A is performed after the aligning step S94. A plurality of cell tissues T can be obtained in such a manner that the extending directions thereof are aligned with each other by dissolving holding material 5A after the plurality of blocks 50 are disposed in such a manner that the extending directions of cell tissues T are aligned with each other. Therefore, it is not necessary to align the extending directions of cell tissues T with a pair of tweezers or the like, and thereby the risk of damaging cell tissues T can be reduced. Since the dissolution conditions are the same as those of the first embodiment, the description thereof will not be repeated.

Preferably, when block 50 are disposed on net 200 having a wavy structure and then dissolved, cell tissues T can be collected in recess 220, and thereby a bundle of cell tissues T can be more easily obtained in such a manner that the extending directions thereof are aligned with each other.

### THIRD EMBODIMENT

In the third embodiment, holding material 5A is housed in a dividing housing provided with a plurality of blades, and then holding material 5A is dissolved in the dividing housing to collect the cell tissues.

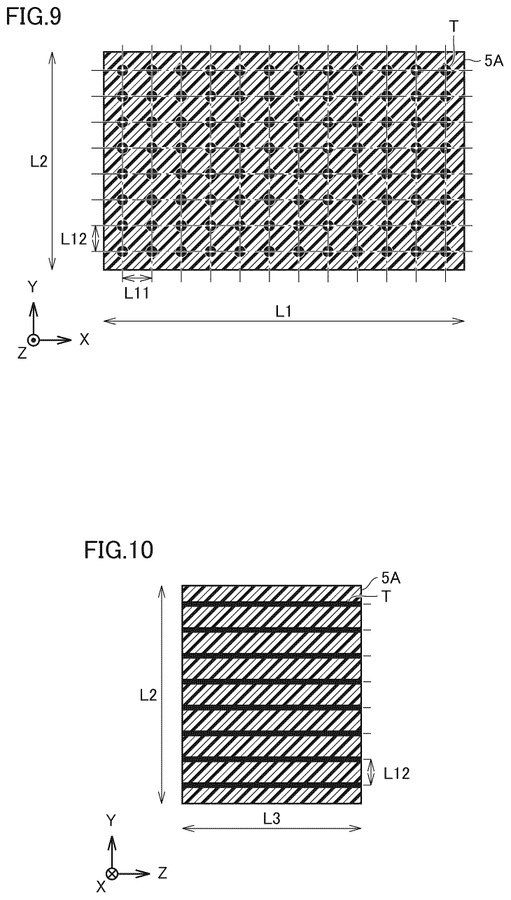

The production method according to the third embodiment is the same as the production method illustrated in FIGS. 1 and 2 up to the separating step S8. FIGS. 9 and 10 illustrate holding material 5A after the separating step S8 according to the third embodiment.

In FIG. 9 and subsequent figures, the extending direction of cell tissues T held by holding material 5A is defined as a Z-axis direction, and a plane orthogonal to the Z-axis direction is defined as an XY plane. In one example, in the dissolving step, the vertically upward direction is defined as the Y-axis positive direction, and the vertically downward direction is defined as the Y-axis negative direction. In FIG. 9 and subsequent figures, the right direction may be defined as the X-axis positive direction, and the left direction may be defined as the X-axis negative direction. In addition, the positive direction of the Y axis may be defined as the upward direction, and the negative direction of the Y axis may be defined as the downward direction. The front direction may be defined as the Z-axis positive direction, and the rear direction may be defined as the Z-axis negative direction. Further, the length in the X-axis direction may be defined as the width, and the length in the Z-axis direction may be defined as the depth.

FIG. 9 is a view of holding material 5A viewed from the Z-axis direction. FIG. 10 is a view of holding material 5A viewed from the X-axis direction.

In the examples of FIGS. 9 and 10, cell tissues T in holding material 5A are arranged at equal intervals in parallel to each other. The length of holding material 5A along the X-axis direction is denoted by L1, the length of holding material 5A along the Y-axis direction is denoted by L2, and the length of holding material 5A along the Z-axis direction is denoted by L3. An interval of holding material 5A along the X-axis direction is defined as L11, and an interval thereof along the Y-axis direction is defined as L12.

In one example of the third embodiment, holding material 5A that contains cell tissues T is prepared as follows. First, a dispenser equipped with six syringes is prepared, and the six syringes are spaced apart from each other at intervals of 9 mm in correspondence to a 96-well plate having a well array of 8×12 wells (at intervals of 9 mm), each of which contains a bio-ink containing cells such as muscles. The feed amount of the dispenser is set to 4.5 mm in both the X direction and the Y direction so as to print cells C with a fiber length of 20 mm in twelve rows, and each row is printed with 8 lines of cells C. Then, after cell tissues T are obtained by culturing cells C, the periphery of cell tissues T is replaced with gelatin. By cooling gelatin to 4° C. to cure the same, cell tissues T can be fixed in gelatin. In one example, the size of the cured gelatin may be 54 mm×36 mm×20 mm. In other words, in the present embodiment, L1, L2, L3, L11, and L12 in FIGS. 9 and 10 are 54 mm, 36 mm, 20 mm, 4.5 mm, and 4.5 mm, respectively.

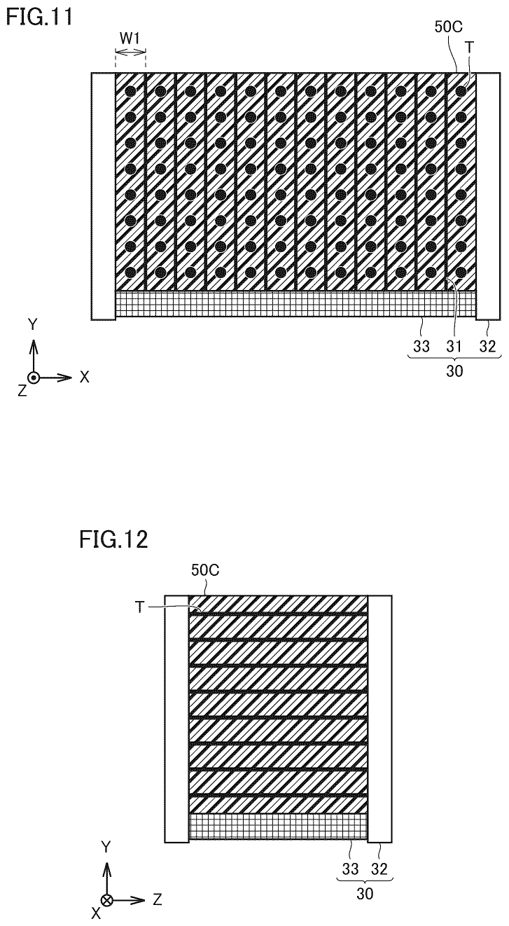

FIGS. 11 and 12 are diagrams illustrating a dividing step S92C according to the third embodiment. In the dividing step S92C, holding material 5A illustrated in FIGS. 9 and 10 is housed in a dividing housing 30, and thereby holding material 5A is divided into a plurality of blocks 50C. FIG. 11 is a view illustrating dividing housing 30 that houses holding material 5A as viewed from the Z-axis direction, and FIG. 12 is a view illustrating dividing housing 30 that houses holding material 5A as viewed from the X-axis direction.

Dividing housing 30 includes a plurality of blades 31 for dividing cured holding material 5A, a side wall 32, and a bottom surface 33. Dividing housing 30 is typically a rectangular parallelepiped housing without an upper surface. Dividing housing 30 may be provided with an upper surface, but in this case, it is preferable to provide a hole so as to allow holding material dissolving liquid 36 to pass through the upper surface of dividing housing 30.

Each of the plurality of blades 31 is a partition plate for creating a plurality of blocks 50C for storage. In the dividing step S92C, cured holding material 5A is pushed into dividing housing 30, and thereby holding material 5A is divided by the plurality of blades 31. As described above, each blade 31 is only required to cut cured holding material 5A, and thereby it is preferable that each blade is formed of a material harder than the cured holding material, but it is not necessary to sharpen each blade. By using dividing housing 30 which is equipped with the plurality of blades 31 arranged with an interval W1, a plurality of blocks 50C can be obtained with a width W1.

In one example, when the holding material (for example, gelatin) is dissolved by heat in the dissolving step, dividing housing 30 is preferably formed of a material (for example, metal) having high thermal conductivity.

At least one of the plurality of blocks 50C contains at least one cell tissue T. The number of cell tissues T contained in each block 50C may be appropriately selected by a person skilled in the art, and is determined according to the subsequent processing steps, for example. The number of blocks 50C may be two or more. For example, when a plurality of cell tissues T are printed in two directions orthogonal to each other as illustrated in FIG. 9, dividing housing 30 may be configured to divide holding material 5A into one row or two or more rows.

In the examples of FIGS. 11 and 12, a dividing housing 30 provided with a plurality of blades arranged with an interval W1 is used such that each block 50C contains a single row of cell tissues T. In other words, the blade interval W1 is designed to be the same as the interval L11 of cell tissue T, for example. This results in a plurality of blocks 50C, each of which contains a single row of cell tissue.

In one example, holding material 5A is pushed into dividing housing 30 in parallel with the extending direction (Z-axis direction) of cell tissue T. In this case, the front wall or the rear wall of dividing housing 30 is preferably formed as an openable and closable lid. As one example, the lid of dividing housing 30 is configured to be removable. Accordingly, after the lid of dividing housing 30 is opened and holding material 5A is pushed into dividing housing 30, the lid is closed, which makes it possible to prevent the position of cell tissue T separated from holding material 5A from being shifted in the Z-axis direction. Thus, it is possible to prevent cell tissue T from protruding or falling from bottom surface 33.

In one example, the rear wall is configured as a lid, and the thickness of the lid is different from the thickness of the front wall. For example, if the thickness of the lid is made thicker than that of the front wall, a front portion of each block 50C, which is more susceptible to heat transfer, will dissolve faster than a rear portion thereof. As a result, cell tissues T in the rear portion may sag, bend, or stick to the blade or the side wall. Therefore, the thickness of the lid is preferably the same as the thickness of the front wall. Alternatively, as will be described later regarding a collection housing 40 in the fourth embodiment, it is preferable that the length of dividing housing 30 in the Z-axis direction is configured to be longer than the length of block 50C, and a gap is provided between block 50C and dividing housing 30.

In another embodiment, holding material 5A is pushed in parallel to the Y-axis direction of dividing housing 30.

Preferably, a lower portion of dividing housing 30 is provided with a hole for discharging the dissolved holding material 5A. The hole is configured not to allow cell tissue T to pass therethrough. For example, at least one hole is formed in a lower portion of side wall 32 and/or bottom surface 33. More preferably, at least one hole is formed between adjacent blades. Further preferably, bottom surface 33 is formed into a mesh that includes a plurality of holes. The mesh is, for example, 100-mesh (a mesh in which 100×100 threads are woven into a square having a side length of 25.4 mm). This allows the dissolved holding material 5A to pass through the holes of bottom surface 33, and allows cell tissue T separated by holding material 5A to be held on bottom surface 33 in parallel with bottom surface 33. In addition, as the number of holes becomes numerous and widely distributed as in a mesh, the dissolved holding material 5A can pass through a wide area of bottom surface 33, resulting in faster and more uniform dissolution.

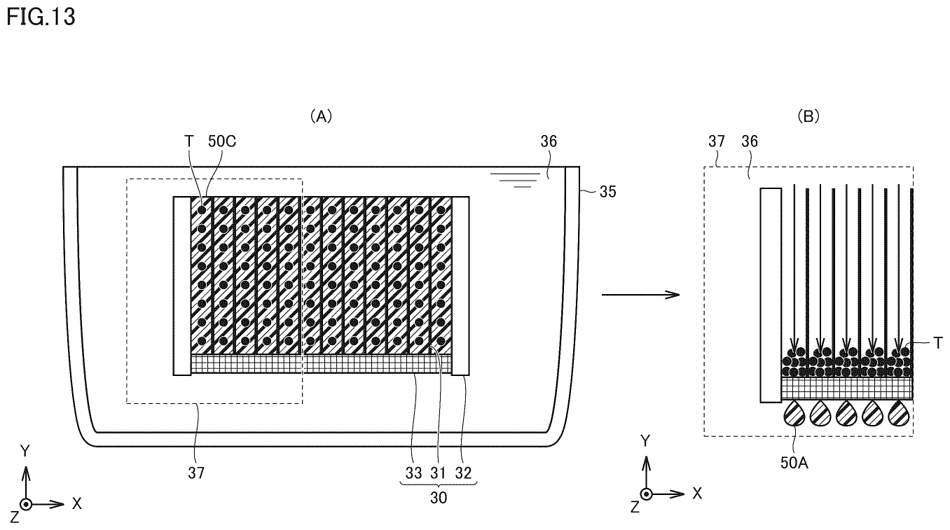

FIG. 13 is a diagram illustrating a dissolving step S9C according to the third embodiment.

The dissolving step S9C is performed after the dividing step S92C. FIG. 13A illustrates a state in which dividing housing 30 that houses the plurality of blocks 50C is immersed in holding material dissolving liquid 36 contained in a bath 35. FIG. 13B illustrates a state in which holding material 5A of each block 50C is dissolved by immersing dividing housing 30 in holding material dissolving liquid 36. FIG. 13B only illustrates a part 37 surrounded by a dashed line in FIG. 13A.

As illustrated in FIG. 13, by immersing dividing housing 30 in holding material dissolving liquid 36, holding material 5A of the plurality of blocks 50C housed in dividing housing 30 is dissolved and discharged to the outside of dividing housing 30. Thus, cell tissues T are aligned between adjacent blades 31 or between blade 31 and side wall 32. Accordingly, the plurality of cell tissues T can be collected in such a manner that the extending directions are aligned with each other.

The dissolution conditions of the third embodiment are the same as those of the first embodiment.

Holding material dissolving liquid 36 preferably has a specific gravity smaller than that of holding material 5A. Thus, holding material 5A dissolved in dividing housing 30 can pass through the hole in a lower portion of dividing housing 30 by its own weight. Therefore, the dissolution rate of blocks 50C in dividing housing 30 can be increased and uniformized.

Holding material dissolving liquid 36 preferably has a specific gravity smaller than that of cell tissue T. Thus, cell tissue T separated from holding material 5A in dividing housing 30 sinks to bottom surface 33 by its own weight. Therefore, it is possible to prevent cell tissues T from becoming non-parallel to bottom surface 33 between adjacent blades 31 in dividing housing 30. The state in which cell tissues T become non-parallel to bottom surface 33 means that at least a part of cell tissues T becomes non-parallel to bottom surface 33 due to the reason of bending, twisting, clinging to each other, or sticking to blades 31 and/or side wall 32, for example.

In one example, holding material 5A is gelatin, holding material dissolving liquid 36 is warm water, and bath 35 is a thermostatic bath. According to this configuration, cell tissues T can be safely collected without any damage.

If bottom surface 33 of dividing housing 30 is configured to be detachable from side wall 32, it is possible to easily collect cell tissues T without disrupting the state of cell tissues T aligned on bottom surface 33.

As described above, according to the third embodiment, holding material 5A can be easily divided by housing holding material 5A in dividing housing 30 which is provided with the plurality of blades 31. The aligned cell tissues T can be easily obtained simply by immersing dividing housing 30 in holding material dissolving liquid 36. Therefore, different from the first embodiment, it is not necessary to align cell tissues T with a pair of tweezers or the like. In addition, as compared with the second embodiment, since the aligning step S94 of aligning the plurality of blocks 50 is not performed, it is possible to save time and effort. In particular, according to the third embodiment, it is not possible for the user to mistakenly arrange the plurality of blocks 50 so that the extending direction of recess 220 is not aligned with the extending direction of cell tissues T included in the plurality of blocks 50 as in the aligning step S94 according to the second embodiment. Therefore, cell tissues T can be collected more easily and accurately than in the first embodiment and the second embodiment, with the extending directions thereof aligned with each other.

### FOURTH EMBODIMENT

In the fourth embodiment, after holding material 5A is divided into a plurality of blocks, the plurality of blocks are spaced apart from each other, and then holding material 5A is dissolved.

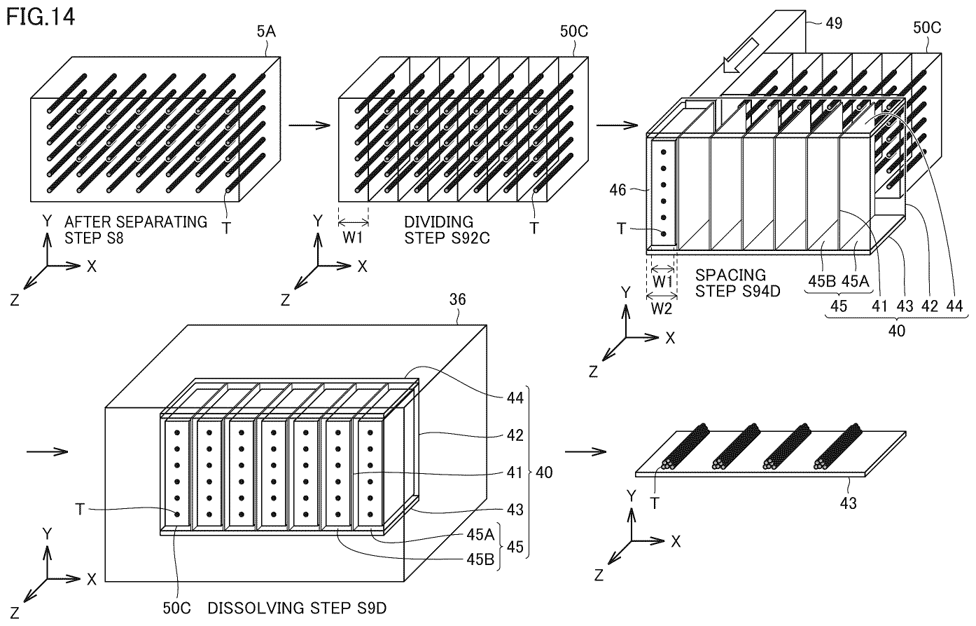

FIG. 14 is a schematic diagram illustrating a production method according to the fourth embodiment. The production method according to the fourth embodiment is the same as the production method according to the third embodiment in the steps up to the dividing step S92C.

With reference to FIG. 14, holding material 5A that contains cell tissues T after the separating step S8 is divided into a plurality of blocks 50C in the dividing step S92C.

In a spacing step S94D according to the fourth embodiment, the plurality of blocks 50C are spaced apart from each other. The spacing step S94D is performed after the dividing step S92C and before the dissolving step S9D.

In the spacing step S94D, the plurality of blocks 50C are spacing apart from each other by a predetermined spacing unit. In the spacing step S94D illustrated in FIG. 14, the plurality of blocks 50C are spaced apart from each other using a collection housing 40.

Collection housing 40 includes a partition 41, a side wall 42, a bottom surface 43, and an upper surface 44. It is preferable that a hole through which holding material dissolving liquid 36 and dissolved holding material 5A pass is provided in an upper portion and a lower portion of collection housing 40, respectively. In one example, at least a portion of bottom surface 43 and upper surface 44 is constituted by a mesh. In another example, the hole provided in an upper portion of collection housing 40 may be a gap provided between at least one side wall 42 and upper surface 44. Alternatively, the upper surface of collection housing 40 may be opened.

Collection housing 40 has a plurality of chambers 45. The plurality of chambers 45 include a chamber 45A enclosed by bottom surface 43, side wall 42 and an adjacent partition 41 and a chamber 45B enclosed by bottom surface 43 and two adjacent partitions 41. Each of the plurality of chambers 45 has a width W2 that is wider than interval W1 between blades 31 that divide holding material 5A in the dividing step S92C of obtaining the plurality of blocks 50C. Width W2 is an interval between side wall 42 and adjacent partition 41 in chamber 45A, and is equal to an interval between two adjacent partitions 41 in chamber 45B. As described above, width W2 of chamber 45 is larger than width W1 of each block 50C to be inserted. Thus, when each block 50C is inserted into each chamber 45, a gap 46 is formed between each block 50C and partition 41 or side wall 42.

In one example, each block 50C is formed of gelatin that contains cell tissue T into a dimension having a width of 4.5 mm and a depth of 20 mm, and collection housing 40 is provided with partitions, each of which has a depth of 28 mm and is separated from each other with an interval of 6.5 mm. Accordingly, a gap of 1 mm in the left-right direction and a gap of 4 mm in the front-rear direction can be formed between each block 50C and side wall 42 of collection housing 40.

When the holding material (for example, gelatin) is dissolved by heat in the dissolving step S9D described later, collection housing 40 is preferably formed of a material (for example, metal) having high thermal conductivity.

In the spacing step S94D of FIG. 14, the plurality of blocks 50C are spaced apart from each other by housing the plurality of blocks 50C in collection housing 40 provided with a plurality of chambers 45 having width W2. In one example, the plurality of blocks 50C are inserted into collection housing 40 in parallel with the extending direction (Z-axis direction) of cell tissue T. In this case, the front wall or the rear wall of collection housing 40 is preferably formed as an openable and closable lid. As one example, the lid of collection housing 40 is configured to be removable. Accordingly, after the lid of collection housing 40 is opened and block 50C is inserted into collection housing 40, the lid is closed, which makes it possible to prevent the position of cell tissue T separated from holding material 5A from being shifted in the Z-axis direction. Thus, it is possible to prevent cell tissue T from protruding or falling from bottom surface 43.

In one example, the insertion of each block 50C into each chamber 45 is performed using a pusher 49 having approximately the same width as each block 50C. Pusher 49 may be controlled by a controller (not shown) or may be manually moved by a user. As described above, the plurality of blocks 50C may be easily spaced apart from each other by using collection housing 40. Collection housing 40 and pusher 49 correspond to an embodiment of a “spacing unit”.

In the dissolving step S9D of FIG. 14, cell tissues T can be easily collected by immersing collection housing 40 that houses blocks 50C in holding material dissolving liquid 36 so as to dissolve holding material 5A. In the dissolving step S9D of FIG. 14, holding material dissolving liquid 36 enters gap 46 between each block 50 and the partition 41 or side wall 42, and thereby, holding material dissolving liquid 36 also comes into contact with the side surface of each block 50 (the surface facing partition 41 or side wall 42). The portion of holding material 5A that comes into contact with holding material dissolving liquid 36 dissolves faster than the portion thereof that does not come into contact with holding material dissolving liquid 36. The portion of holding material 5A that does not come into contact with holding material dissolving liquid 36 includes, for example, a portion of holding material 5A that comes into contact with partition 41 or side wall 42, and a portion inside holding material 5A. As described above, as compared with the third embodiment in which the plurality of blocks 50C are not spaced apart from each other (in other words, there is no gap between each block 50 and partition 41 or side wall 42), holding material 5A of each block 50 can be dissolved rapidly and uniformly. In particular, according to the fourth embodiment, it is possible to rapidly dissolve a part of the plurality of blocks 50C located in a central portion of dividing housing 30, which is considered to be relatively difficult to transfer heat and thereby is relatively difficult to dissolve in the third embodiment. As described above, by spacing the plurality of blocks 50C apart from each other, it is possible to collect the cell tissues more rapidly, and it is also possible to obtain a bundle of cell tissues T with the extending directions thereof aligned with each other.

In the present specification, the expression that “a plurality of blocks are not spaced apart from each other” includes not only a case where the plurality of blocks are arranged in direct contact with each other without any gap therebetween, but also a case where the plurality of blocks are arranged in indirect contact with each other with a blade inserted therebetween as in the third embodiment. More specifically, the expression that “a plurality of blocks are not spaced apart from each other” means that cross sections obtained by dividing cured holding material 5A are not spaced apart from each other.

The mode of spacing the plurality of blocks in the spacing step S94D is not limited to the example of FIG. 8, and for example, in the dividing step S92C, the plurality of blocks may be spaced apart from each other by cutting off a portion of each block that does not include cell tissue T so as to reduce the width of each block. In the second embodiment (FIG. 8) described above, the plurality of blocks divided in the dividing step S92 are arranged at spaced positions in the spacing step of spacing the plurality of blocks.

As described above, according to the production method according to the fourth embodiment, the plurality of blocks 50C can be rapidly and uniformly dissolved by spacing the plurality of blocks 50C apart from each other, and thereby cell tissues T can be rapidly collected in an aligned state. In particular, according to the example of FIG. 14, the plurality of blocks 50C can be easily and reliably spaced apart from each other by using collection housing 40.

Although in the above it is mainly described that the plurality of blocks 50C are spaced apart from each other along the X-axis direction, if the plurality of blocks 50C are also spaced apart from side wall 32 in the Z-axis direction, the dissolution rate of the plurality of blocks 50C can be further increased. In other words, if a gap is formed between each of the front wall and the rear wall and each block 50C, the dissolution efficiency can be improved. In addition, according to this configuration, even in a case where the thickness of the lid (for example, the rear wall) and the thickness of a side wall (for example, the front wall) that faces the lid are different from each other in collection housing 40, it is possible to make the dissolution rate of holding material 5A in the vicinity of the lid substantially equal to the dissolution rate of holding material 5A in the vicinity of the side wall that faces the lid.

### Modification of the Fourth Embodiment

In a modification of the fourth embodiment, the plurality of blocks are housed in the collection housing in such a manner that each of the plurality of blocks is not housed in adjacent chambers. Hereinafter, the modification according to the fourth embodiment is also simply referred to as the modification. In the production method according to the modification, the steps up to the dividing step S92C are the same as those in the production method according to the third embodiment and the production method according to the fourth embodiment.

The spacing step S94Da according to the modification is performed after the dividing step S92C and before the dissolving step S9Da.

In the spacing step S94Da, the plurality of blocks 50C are housed in a collection housing 40a in such a manner that each of the plurality of blocks is not housed in adjacent chambers.

Collection housing 40a corresponds to an embodiment of a “spacing unit”. Collection housing 40a may be configured in the same manner as collection housing 40, but in one example, it is different from collection housing 40 in that width W3 of chamber 45 is configured to be equal to interval W1 between the blades that divide holding material 5A in the dividing step S92C (which will be described in detail hereinafter). Side wall 42a of collection housing 40a includes a side wall 42Aa on the Z-axis positive direction, a side wall 42Ba on the Z-axis negative direction, and a side wall 42Ca horizontal to the YZ plane. In one example, side wall 42Aa is formed as an openable and closable lid. Thus, after the lid is opened and block 50C is inserted, the lid is closed, which makes it possible to prevent the position of cell tissue T separated from holding material 5A from being shifted in the Z-axis direction. Thus, it is possible to prevent cell tissue T from protruding or falling from bottom surface 43a.

Collection housing 40a includes a plurality of chambers 45a. The plurality of chambers 45a include a chamber 45Aa enclosed by bottom surface 43a, an adjacent partition 41a and side wall 42Ca, and a chamber 45Ba enclosed by bottom surface 43a and two adjacent partitions 41a.

The spacing step S94Da is performed in substantially the same manner as the spacing step S94D, but in the spacing step S94Da, after one block 50C is inserted into one chamber 45a, a next block 50C is inserted into a next chamber 45a with at least one empty chamber 45a interposed therebetween. Thus, in collection housing 40a, the plurality of blocks 50C are stored in chambers with at least one empty chamber 45a interposed therebetween. Therefore, chamber 45a on each side of one chamber 45a which is inserted with one block 50C is an empty chamber in which no block 50C is inserted.

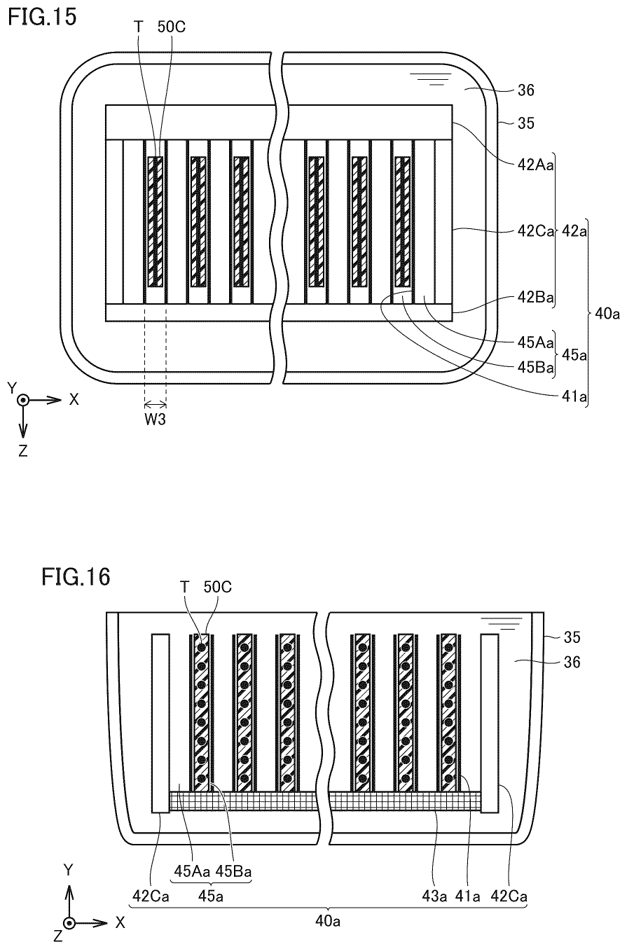

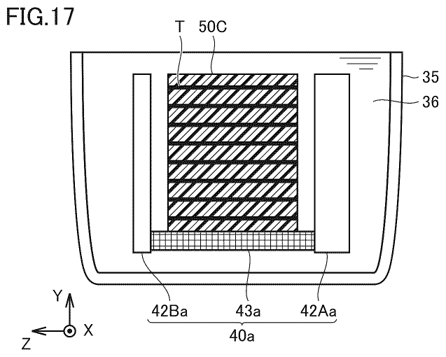

In one example, in the spacing step S94Da, as illustrated in the following FIGS. 15 to 17, each block 50C is housed in one chamber 45a followed with an empty chamber. As a result, the plurality of blocks 50C can be most efficiently inserted when both chambers adjacent to the chamber inserted with one block 50C are made empty. In other words, the largest number of blocks 50C can be stored in a state in which both adjacent chambers are made empty.

FIGS. 15 to 17 are diagrams illustrating the dissolving step S9Da according to the modification. In the dissolving step S9Da of FIGS. 15 to 17, collection housing 40a that houses the plurality of blocks 50C inserted with at least one empty chamber 45a therebetween is immersed in holding material dissolving liquid 36. Accordingly, in one example, even if width W3 of each chamber is equal to width W1 of each block 50C and thereby no gap is present between each block 50C and partition 41a or side wall 42Ca, heat can be transferred to the side surfaces of each block 50C via partition 41a and side wall 42Ca, and thereby holding material 5A can be uniformly dissolved by heat. In addition, block 50C located closer to the center of collection housing 40a can be dissolved at the same speed as block 50C located further outside collection housing 40a.

However, in collection housing 40a according to one example, when side wall 42Ca, which requires a greater strength than partition 41A, is made thicker than partition 41a, it takes longer time to dissolve holding material 5A in block 50C inserted into chamber 45Ba as compared to that in block 50C inserted into chamber 45Aa. In this case, it is preferable not to insert block 50C into chamber 45Ba.

In one example, when the dissolving step S9Da according to the modification was performed using collection housing 40a having the same configuration as dividing housing 30 according to the second embodiment, the dissolving step, which would take about 20 minutes in the second embodiment, could be shortened to about 6 minutes. The period of time required for the dissolving step is counted from a time when the housing that houses the plurality of blocks 50C is immersed in holding material dissolving liquid 36 to dissolve holding material 5A to a time when holding material 5A in all of the plurality of blocks 50C is completely dissolved.

In the dissolving step S9D according to the fourth embodiment described above, in order to space the plurality of blocks 50C apart from each other, it is necessary to dispose each block 50C in each chamber 45 in such a manner that a gap is formed between each block 50C and partition 41. On the other hand, according to the dissolving step S9Da of the modification, the plurality of blocks 50C can be reliably spaced apart from each other simply by housing the plurality of blocks 50C in chambers 45a with at least one empty chamber interposed therebetween. However, in the dissolving step S9D according to the fourth embodiment, if each block 50C is disposed in such a manner that a gap is formed between each block 50C and the partition, holding material dissolving liquid 36 can be brought into contact with the side surface of each block 50C instead of the partition, which increases the dissolving rate of each block 50C.

Further, also in collection housing 40a according to the modification, similarly to collection housing 40 according to the fourth embodiment, the width of chamber 45a may be formed larger than the width of each block 50C so as to form a gap between each block 50C and partition 41a. Thus, the dissolution rate of each block 50C can be further increased.

In addition, in collection housing 40a, it is preferable that each of rear side wall 42Aa and front side wall 42Ba is separated from each block 50C. Thus, the dissolution rate of each block 50C can be further increased. As illustrated in FIG. 15, even in an example in which the thickness of the lid (side wall 42Aa) is different from the thickness of a side wall (side wall 42Ba) that faces the lid, the dissolution rate of holding material 5A in the vicinity of the lid can be made equal to the dissolution rate of holding material 5A in the vicinity of the side wall that faces the lid.

As described above, according to the first to fourth embodiments and the modifications, it is possible to provide a production method and a culture device of a cell tissue that can prevent damage to the cell tissue and have good production efficiency. In particular, according to the second to fourth embodiments and the modifications, by dividing block 50C into a desired size and then dissolving the same, it is possible to obtain the divided cell tissues T whose extending directions are aligned with each other. For example, when each block 50C is divided into equal parts, cell tissues T can be obtained at small bundles. This eliminates the need for a conventional step of aligning cell tissues T having different extending directions using a pair of tweezers, thereby significantly reducing the work time. In addition, it is not necessary to divide cell tissues T into small bundles. Further, according to the third embodiment to the fourth embodiment and the modifications thereof in particular, since the alignment step can be omitted as compared with the second embodiment, it is possible to obtain a bundle of cell tissues T in which the extending directions are aligned more accurately and easily. Further, according to the fourth embodiment and the modification thereof in particular, since blocks 50C can be dissolved more uniformly and rapidly than the third embodiment, the time required for the dissolving step can be shortened.

### Aspects

It will be understood by those skilled in the art that the embodiments described above are specific examples of the following aspects.

(First Aspect) The production method according to one aspect is a production method of a plurality of cell tissues. The production method includes: linearly printing a plurality of cells between a first substrate and a second substrate in such a manner that both ends of the plurality of cells are supported on the first substrate and the second substrate, respectively; obtaining the plurality of cell tissues by culturing the printed plurality of cells; filling, between the first substrate and the second substrate, a soluble liquid holding material that cures under a predetermined condition; curing the filled holding material; separating each of the plurality of cell tissues from at least one of the first substrate and the second substrate by cutting out the cured holding material from at least one of the first substrate and the second substrate; and obtaining a plurality of blocks by dividing the cured holding material along an extending direction of the plurality of cell tissues held by the cured holding material. The obtaining of the plurality of blocks includes housing the cured holding material in a dividing housing which is provided with a plurality of blades and is configured to divide and house the cured holding material.

According to the production method described in the first aspect, by separating the cell tissues from the first substrate and the second substrate with the cell tissues held by the cured holding material, it is possible to prevent damage to the cell tissues and improve the collection efficiency of the cell tissues. In addition, since the cell tissues can be handled in each block, and since the cell tissues are protected by the cured holding material constituting the blocks, the cell tissues can be easily handled. In addition, in a case where a combination operation of combining a plurality of types of cell tissues is performed to create an aggregate that mimics the structure of a specific tissue, it is easier to perform the combination operation by performing the combination operation in each block than in a case where the combination operation is performed on each cell tissue. Further, the use of a dividing housing makes it easy to divide the holding material. Therefore, it is possible to provide a production method that prevents damage to the cell tissue and improves the production efficiency.

(Second Aspect) The production method described in the first aspect further includes collecting the plurality of cell tissues by dissolving the holding material that constitutes each of the plurality of blocks housed in the dividing housing.

According to the production method described in the second aspect, it is possible to easily obtain the cell tissues aligned with each other.

(Third Aspect) The production method according to the first or second aspect further includes spacing the plurality of blocks apart from each other.

According to the production method described in the third aspect, it is possible to collect the cell tissues more rapidly, and it is possible to obtain a bundle of cell tissues with the extending directions thereof aligned with each other.

(Fourth Aspect) In the production method according to the third aspect, the spacing of the plurality of blocks apart from each other includes housing each of the plurality of blocks in a collection housing that includes a plurality of chambers having a width wider than an interval between adjacent blades that divide the holding material in the obtaining of the plurality of blocks.

According to the production method described in the fourth aspect, a plurality of blocks can be easily and reliably spaced apart from each other.

(Fifth Aspect) In the production method according to the third or fourth aspect, the spacing of the plurality of blocks apart from each other includes housing the plurality of blocks in a collection housing that includes a plurality of chambers in such a manner that each of the plurality of blocks is not housed in chambers adjacent to each other.

According to the production method described in the fifth aspect, it is easy to space the plurality of blocks apart from each other simply by housing the plurality of blocks in chambers with at least one empty chamber interposed therebetween.

(Sixth Aspect) The culture device according to one aspect is a bioprinting culture device for culturing a plurality of cells to obtain a plurality of cell tissues. The culture device includes: a culture housing in which a first chamber with a first substrate for supporting the plurality of cells disposed therein, a storage chamber configured to store a culture solution, and a second chamber with a second substrate for supporting the plurality of cells disposed therein are provided in this order; a replacing unit that replaces the solution stored in the storage chamber with a soluble liquid holding material that cures under a predetermined condition; and a separating unit. The plurality of cells are linearly printed from the first chamber toward the second chamber. The separating unit is configured to separate each of the plurality of cell tissues stored in the storage chamber from at least one of the first substrate disposed in the first chamber and the second substrate disposed in the second chamber. The culture device further includes a dividing unit that divides the holding material along an extending direction of the plurality of cell tissues held by the holding material to obtain a plurality of blocks.

According to the culture device described in the sixth aspect, since the holding material that is cured under a predetermined condition can be inserted into the storage chamber by using the replacing unit, the cell tissues can be separated from the substrate while each of the cell tissues is being held by the cured holding material. This makes it possible to prevent damage to the cell tissues and improve the collection efficiency of the cell tissues. In addition, since the cell tissues can be handled in each block, and since the cell tissues are protected by the cured holding material constituting each block, the cell tissues can be easily handled. In addition, in a case where a combination operation of combining a plurality of types of cell tissues is performed to create an aggregate that mimics the structure of a specific tissue, it is easier to perform the combination operation by performing the combination operation in each block than in a case where the combination operation is performed on each cell tissue. Further, the use of a dividing housing makes it easy to divide the holding material. Therefore, it is possible to provide a production device that prevents damage to the cell tissue and improves the production efficiency.

(Seventh Aspect) The culture device according to the sixth aspect further includes a dissolving unit that dissolves the holding material to collect the plurality of cell tissues.

According to the culture device described in the seventh aspect, the cell tissues can be collected simply by dissolving the separated holding material.

(Eighth Aspect) The culture device according to item 6 or item 7 further includes a spacing unit that spaces the plurality of blocks apart from each other.

According to the culture device described in the eighth aspect, it is possible to collect the cell tissues more rapidly, and it is possible to obtain a bundle of cell tissues with the extending directions thereof aligned with each other.

It is contemplated that the embodiments disclosed herein may be appropriately combined and implemented as long as they are not technically contradictory to each other. It should be understood that the embodiments disclosed herein are illustrative and non-restrictive in all respects. The scope of the present invention is defined by the terms of the claims rather than the description of the embodiments above, and is intended to include any modifications within the scope and meaning equivalent to the terms of the claims.

It should be understood that the embodiments disclosed herein have been presented for the purpose of illustration and description but not limited in all aspects. It is intended that the scope of the present invention is not limited to the description above but defined by the scope of the claims and encompasses all modifications equivalent in meaning and scope to the claims.

What is claimed is:

1. A production method of a plurality of cell tissues, comprising:
linearly printing a plurality of cells between a first substrate and a second substrate in such a manner that both ends of the plurality of cells are supported on the first substrate and the second substrate, respectively;
obtaining the plurality of cell tissues by culturing the printed plurality of cells;
filling, between the first substrate and the second substrate, a soluble liquid holding material that cures under a predetermined condition;
curing the filled holding material;
separating each of the plurality of cell tissues from at least one of the first substrate and the second substrate by cutting out the cured holding material from at least one of the first substrate and the second substrate; and
obtaining a plurality of blocks by dividing the cured holding material along an extending direction of the plurality of cell tissues held by the cured holding material,
the obtaining of the plurality of blocks including housing the cured holding material in a dividing housing which is provided with a plurality of blades and is configured to divide and house the cured holding material.

2. The production method according to claim 1, further comprising:
collecting the plurality of cell tissues by dissolving the holding material that constitutes each of the plurality of blocks housed in the dividing housing.

3. The production method according to claim 1, further comprising:
spacing the plurality of blocks apart from each other.

4. The production method according to claim 3, wherein
the spacing of the plurality of blocks apart from each other includes housing each of the plurality of blocks in a collection housing that includes a plurality of chambers having a width wider than an interval between adjacent blades that divide the holding material in the obtaining of the plurality of blocks.

5. The production method according to claim 3, wherein
the spacing of the plurality of blocks apart from each other includes housing the plurality of blocks in a collection housing that includes a plurality of chambers in such a manner that each of the plurality of blocks is not housed in chambers adjacent to each other.

6. A bioprinting culture device for culturing a plurality of cells to obtain a plurality of cell tissues, the culture device comprising:
a culture housing in which a first chamber with a first substrate for supporting the plurality of cells disposed therein, a storage chamber configured to store a culture solution, and a second chamber with a second substrate for supporting the plurality of cells disposed therein are provided in this order;
a replacing unit that replaces the solution stored in the storage chamber with a soluble liquid holding material that cures under a predetermined condition; and
a separating unit,
the plurality of cells being linearly printed from the first chamber toward the second chamber,
the separating unit being configured to separate each of the plurality of cell tissues stored in the storage chamber from at least one of the first substrate disposed in the first chamber and the second substrate disposed in the second chamber, and
the culture device further comprising:
a dividing unit that divides the holding material along an extending direction of the plurality of cell tissues held by the holding material to obtain a plurality of blocks.

7. The culture device according to claim 6, further comprising:
a dissolving unit that dissolves the holding material to collect the plurality of cell tissues.

8. The culture device according to claim 6, further comprising:
a spacing unit that spaces the plurality of blocks apart from each other.
